(19) United States
(12) Patent Application Publication
(10) Pub. No.: US 2025/0276162 A1
(43) Pub. Date: Sep. 4, 2025

(54) METHOD OF REDUCING CATHETER EXCHANGES DURING PERFORMANCE OF A PERCUTANEOUS PORTOSYSTEMIC SHUNT PROCEDURE

(71) Applicants: Jacob Cynamon, Bronx, NY (US); Jonathan Levine, Clarendon Hills, IL (US); Timothy Murphy, Cambridge, MA (US)

(72) Inventors: Jacob Cynamon, Bronx, NY (US); Jonathan Levine, Clarendon Hills, IL (US); Timothy Murphy, Cambridge, MA (US)

(21) Appl. No.: 19/054,988

(22) Filed: Feb. 17, 2025

Related U.S. Application Data
(60) Provisional application No. 63/559,620, filed on Feb. 29, 2024.

(51) Int. Cl. A61M 27/00; A61M 25/10

(52) U.S. Cl. CPC A61M 27/002; A61M 25/104; A61M 2025/1079; A61M 2210/1071

(57) ABSTRACT

The invention comprises a method of performance of a percutaneous portosystemic shunt procedure, said method comprising a means of improving operator efficiency.

### CROSS-REFERENCE TO RELATED APPLICATIONS

This application claims the benefit of U.S. Provisional Patent Application No. 63/559,620, filed Feb. 29, 2024, entitled “METHOD OF REDUCING CATHETER EXCHANGES DURING PERFORMANCE OF A PERCUTANEOUS PORTOSYSTEMIC SHUNT PROCEDURE,” the entire contents of which are incorporated herein by reference.

### REFERENCES

Rossle M. TIPS: 25 years later. Journal of Hepatology 2013 vol. 59:1081-1093

| | Publication/ | |
|---|---|---|
| Patent Number | Issue Date | Patentee |
| U.S. Pat. No. 10, 448,931 | Oct. 22, 2019 | Cynamon |
| U.S. Pat. No. 8,287,481B1 | Oct. 16, 2012 | Kahn |
| U.S. Pat. No. 8,628,491B2 | Jan. 14, 2014 | Kahn |
| U.S. Pat. No. 8,632,468B2 | Jan. 21, 2014 | Glossop |
| U.S. Pat. No. 9,072,596B1 | Jul. 7, 2015 | Kahn |
| US20100217117A1 | Aug. 26, 2010 | Glossop |
| US20130245533A1 | Sep. 19, 2013 | Kahn |
| U.S. Pat. No. 11,504,508B2 | Nov. 22, 2022 | Levine |
| U.S. Pat. No. 4,641,654 | Feb. 10, 1987 | Wilfred J. Samson |
| U.S. Pat. No. 4,842,590 | Jun. 27, 1989 | Susumu Tanabe |
| U.S. Pat. No. 5,087,247 | Feb. 11, 1992 | Josephe B. Horn |
| U.S. Pat. No. 5,328,469 | Jul. 12, 1994 | Roger Coletti |
| U.S. Pat. No. 5,538,510 | Jul. 23, 1996 | Carlos A. Fontirroche |
| U.S. Pat. No. 6,322,577 | Nov. 27, 2001 | Peter R. McInnes |
| U.S. Pat. No. 6,402,720 | Jun. 11, 2002 | Jay Frederic Miller |
| U.S. Pat. No. 10,149,962 | Dec. 11, 2018 | Curtis J. Franklin |
| U.S. Pat. No. 3,382,872 | May 14, 1968 | Melvin L. Rubin |
| | Publication | |
| Patent Appl. No. | Date | Patentee |
| U.S. patent application Ser. No. 16/273,462 | Aug. 13, 2020 | Murphy |
| U.S. patent application Ser. No. 14/800,283 | Jan. 21, 2016 | De Cicco |

### FIELD

The present invention relates to methods for performing surgical procedures, and in particular to methods and devices for performing a shunt procedure between branches of the portal vein and branches of the hepatic vein in the liver.

### BACKGROUND OF THE INVENTION

This description of art is not intended to constitute an admission that any patent, publication, or other information referred to is “prior art” with respect to the invention unless specifically designated as such. In addition, this section should not be construed to mean that a search has been made or that no other pertinent information as defined in 37 C.F.R. § 1.56(a) exists.

Portal hypertension is a medical condition characterized by high blood pressure in the mesenteric or visceral veins in the abdomen, including the portal vein, visceral veins, and their tributaries. Portal hypertension is most often caused by disease of the liver, usually cirrhosis of the liver, but can be caused by any disease that increases flow into the portal circulation, such as for example arteriovenous communication, malformation, or fistula between the arterial circulation and the portal vein or its tributaries, or by restriction of flow of the portal vein caudal to the liver (“pre-hepatic”), within the liver (“hepatic”) or cephalad to the liver (“post-hepatic”).

Portal hypertension results in several medical complications that can be severe and life-threatening, including hemorrhagic complications such as upper gastrointestinal hemorrhage. Upper gastrointestinal hemorrhage associated with portal hypertension is usually caused by dilated veins, called “varices” that try to bypass the diseased liver, but can also be caused for example by portal gastropathy or portal gastropathy caused by portal hypertension.

Most commonly dilated veins originate from the portal or splenic veins, and ascend through the abdomen as coronary or gastric varices and then often into the chest as esophageal varices. Less commonly, varices can be isolated in the stomach, draining in the retroperitoneum through a spontaneous venous connection to a renal vein, which drains into the vena cava and right atrium without having to go through the diseased liver or esophageal veins. In addition to hemorrhage, elevated pressures in the visceral or mesenteric veins can lead to another morbidity of portal hypertension, namely excess accumulation of fluid within the abdomen (“ascites”), or when this fluid can get access to the chest cavity through defects in the diaphragm, pleural effusions or fluid around the lungs (“hydrothorax”).

One way to treat the medical complications of portal hypertension is to create a shunt or bypass within the liver substance that allows intestinal blood to bypass the liver parenchyma and flow through the shunt directly into the draining liver veins and into the right atrium. Open surgical methods to place such a bypass are associated with much higher morbidity than percutaneous or “through the skin” methods that are more commonly used. The percutaneous method of creating a liver shunt is called “transjugular intrahepatic portosystemic shunt”, or TIPS. A TIPS is an extra-anatomic veno-venous bypass.

The transjugular intrahepatic portosystemic shunt (TIPS) was first formed by Dr. Joseph Rosch in 1968 in a dog model. After introduction of metal stents, Dr. Julio Palmaz used stents to improve the TIPS procedure in 1985, and Dr. Goetz Richter performed the first TIPS in a human in 1988. Since then TIPS has become widespread commonly shunts are performed people with portal hypertension and upper gastrointestinal hemorrhage due to esophageal varices, gastric varices, portal gastropathy, refractory ascites or pleural effusion.

An exemplary conventional method to perform the TIPS procedure, done percutaneously with fluoroscopy or other medical imaging guidance, is as follows: access is gained usually into a jugular vein often using Seldinger's method, using a needle and guide wire, then a vascular access sheath is placed into the jugular vein, then through the vascular access sheath a second, longer sheath is introduced over the guidewire and through the vascular access sheath. The second longer sheath is often pre-loaded with a shaped catheter, or once placed a shaped catheter can be placed therethrough. The second longer sheath, catheter and guide wire are then negotiated through the jugular and brachiocephalic veins, superior vena cava, right atrium, intrahepatic inferior vena cava (IVC), and into a hepatic vein, usually the right hepatic vein.

Once access is gained in the hepatic vein, said second longer sheath is advanced distally into the hepatic vein. After removal of the catheter, a second, curved needle is then passed over the guide wire through the second longer sheath, and into the hepatic vein several centimeters caudal to its confluence with the inferior vena cava. The guide wire is then removed, and the needle is unsheathed by retraction of the second larger sheath such that said needle's tip is exposed. After manipulation, the curved needle tip is advanced toward the porta hepatis and portal vein branches, thereby gaining needle access to a portal vein branch.

Once portal vein blood is returned through the needle, a guidewire is advanced into the needle and the portal vein branches peripherally. Then, the second longer sheath is advanced over the curved needle through the parenchyma or tissue of the liver and into a portal vein branch.

Next, the curved needle is removed and usually an angioplasty balloon advanced over the guide wire into the portal vein. This balloon is used to dilate the liver parenchymal tract, with appropriate adjustments in position of the second longer sheath. The balloon then is removed and the outer sheath is again advanced through the tract, and a tube conduit typically introduced over the guide wire into the liver parenchymal tract. The tube conduit is deployed by unsheathing or by retracting the larger outer sheath, and left in the hepatic parenchymal tract. Typically, the tube conduit is left in the liver parenchyma to preserve the portal-vein-to-hepatic vein liver parenchymal tract. Such a tube conduit could be composed of a wire mesh, a textile fabric, xenograft or allograft blood vessel, or combination thereof. This tube conduit comprises the shunt, and functions to connect the portal veins inferior to the liver with the hepatic veins, vena cava, and right atrium superior to the liver, allowing much of the mesenteric blood within the abdomen to bypass the liver parenchyma. Reduced congestion in the mesenteric veins usually relieves the varices, and ascites or pleural effusion.

A person having ordinary skill in the art will readily recognize common variations of this procedure description. For example, in some cases the procedure may be done without the first vascular access sheath, using only the longer access sheath. Also, there may be common differences in the sequence of dilation of the hepatic parenchymal tract, such as dilating the hepatic parenchymal tract before tube conduit placement, dilating the hepatic parenchymal tract after tube conduit placement, or dilating the hepatic parenchymal tract both before and after placement of the tube conduit.

The TIPS procedure is technically challenging to perform and involves many steps and multiple exchanges of needles, guidewires, catheters, and guide sheaths. Because of this, the TIPS procedure is arduous and is considered one of the more challenging procedures done by interventional radiologists.

### SUMMARY

The methods disclosed herein generally optimize the performance of transjugular intrahepatic portosystemic (TIPS) shunts because they reduce the number of steps required to perform the procedure, thereby facilitating the procedure and making the procedure generally more ergonomic to an operator.

An exemplary method and device to address the problem of complexity of the TIPS procedure is by use of a catheter with an inflatable member comprising an extended-tip angioplasty balloon catheter, in one embodiment comprising a distal tapered tip, and another embodiment comprising radiopaque markers. An extended-tip angioplasty balloon catheter is a medical catheter with an inflatable member generally disposed toward a distal half of said medical catheter, with a catheter segment distal to said inflatable member comprising at least 2 cm in length and up to 75 cm in length.

Use of an extended tip angioplasty balloon catheter to perform a TIPS procedure would comprise some of all of the following steps, not necessarily in this order:

1. Obtain percutaneous access to the venous vascular system of a patient; 2. Select a branch of a hepatic vein suitable for performing a TIPS using a catheter and guidewire in combination; 3. Advance a guidesheath over said catheter and guidewire into said hepatic vein; 4. Replace said catheter with a needle; 5. Remove said guidewire and advance said needle through a liver parenchyma into a portal vein; 6. Advance a guidewire through said needle into a portal vein branch; 7. Remove said needle; 8. Advance an extended-tip angioplasty balloon catheter over said guidewire into said portal vein branch so that an extended-tip segment obtains purchase within said portal vein over a secure length, at least 2 cm, prior to said angioplasty balloon entering a liver parenchyma; 9. Optionally, remove said guidewire and replace with a heavier gauge or stiffer working wire; 10. Further advance said extended-tip angioplasty balloon catheter so that an inflatable member is centered within liver parenchyma; 11. Optionally, remove guidewire, perform portal venography through extended tip angioplasty balloon catheter, replace guidewire; 12. Expand said inflatable member to create a pilot lumen within said liver parenchyma; 13. Deflate said inflatable member and retract said extended tip angioplasty balloon catheter into said pilot lumen within said liver parenchyma; 14. Optionally, estimate tract length using marker bands on said extended tip angioplasty balloon catheter when present on a preferred embodiment; 15. Remove said extended tip angioplasty balloon and introduce a conduit, such as a stent or stent-graft, suitable for preserving said pilot lumen in said liver parenchyma; 16. In some embodiments, perform a dilatation of said conduit; 17. Optionally, perform pressure measurements; 18. Optionally, perform portal venography; 19. Remove devices from said patient.

Another embodiment of the invention is:

1. Obtain percutaneous access to the venous vascular system of a patient; 2. Select a branch of a hepatic vein suitable for performing a TIPS using a catheter and guidewire in combination; 3. Advance a guidesheath over said catheter and guidewire into said hepatic vein; 4. Replace said catheter with a needle; 5. Remove said guidewire and advance said needle through a liver parenchyma into a portal vein; 6. Advance a guidewire through said needle into a portal vein branch; 7. Remove said needle; 8. Advance said guidesheath in combination with a dilator over said guidewire into said portal vein; 9. remove said dilator from said patient's body; 10. Advance an extended-tip angioplasty balloon catheter over said guidewire into said portal vein branch so that an extended-tip segment obtains purchase within said portal vein over a secure length, at least 2 cm, prior to said angioplasty balloon entering a liver parenchyma; 9. Optionally, remove said guidewire and replace with a heavier gauge or stiffer working wire; 10. Further advance said extended-tip angioplasty balloon catheter so that an inflatable member is centered within liver parenchyma; 11. Optionally, remove guidewire, perform portal venography through extended tip angioplasty balloon catheter, replace guidewire; 12. Expand said inflatable member to create a pilot lumen within said liver parenchyma; 13. Deflate said inflatable member and retract said extended tip angioplasty balloon catheter into said pilot lumen within said liver parenchyma; 14. Optionally, estimate tract length using marker bands on said extended tip angioplasty balloon catheter when present on a preferred embodiment; 15. Remove said extended tip angioplasty balloon and introduce a conduit, such as a stent or stent-graft, suitable for preserving said pilot lumen in said liver parenchyma; 16. In some embodiments, perform a dilatation of said conduit; 17. Optionally, perform pressure measurements; 18. Optionally, perform portal venography; 19. Remove devices from said patient.

Those skilled in the art will recognize that other embodiments may include injection exit ports in the extended tip segment distal to the balloon to facilitate portal venography.

### BRIEF DESCRIPTION OF THE DRAWINGS

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

FIG. 1 shows an exemplary embodiment of a means of reducing catheter exchanges during performance of a percutaneous portosystemic shunt procedure comprising an angioplasty balloon with extended tip, lateral view.

FIG. 2 illustrates an alternative embodiment of a means of reducing catheter exchanges during performance of a percutaneous portosystemic shunt procedure comprising an angioplasty balloon with extended tip, in this example showing an angled element at the distal end of the extended tip, lateral view.

FIG. 3 is a longitudinal section of an exemplary embodiment of a means of reducing catheter exchanges during performance of a percutaneous portosystemic shunt procedure comprising an angioplasty balloon with an extended tip.

FIG. 4 is a longitudinal section of another representative embodiment of a means of reducing catheter exchanges during performance of a percutaneous portosystemic shunt procedure comprising an angioplasty balloon with an extended tip, in this example also showing marker bands securely mounted in the extended tip.

FIG. 5 is a longitudinal section of another representative embodiment of a means of reducing catheter exchanges during performance of a percutaneous portosystemic shunt procedure comprising an angioplasty balloon with an extended tip, in this example also showing injection exit ports in the extended tip.

FIG. 6 is a longitudinal section view of one embodiment of a means of reducing catheter exchanges during performance of a percutaneous portosystemic shunt procedure comprising an angioplasty balloon with an extended tip, in this example showing an angled distal tip component and side holes in the extended tip.

FIG. 7 is a schematic representation of an anterior-posterior view of an exemplary TIPS procedure illustrating use of an extended-tip angioplasty balloon catheter.

### DETAILED DESCRIPTION

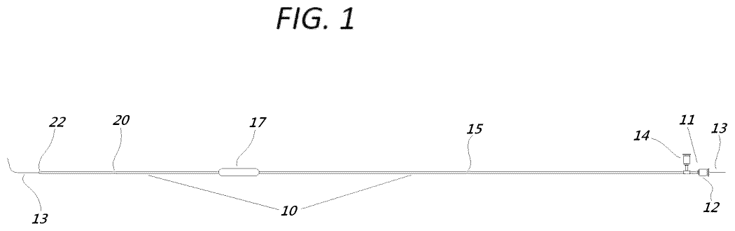

Referring now to FIG. 1, there is shown an examplary embodiment of a means of reducing catheter exchanges during performance of a percutaneous portosystemic shunt procedure that comprises an extended-tip angioplasty balloon catheter 10, wherein said extended-tip angioplasty balloon catheter 10 has at least one lumen to accommodate a guidewire 13, said guidewire port configuration comprising either an Over-the-Wire (OTW) or Rapid Exchange (Rx) design. The catheter 10 comprises a balloon 17 positioned on the shaft of the catheter, a catheter segment 15 proximal to the balloon 17, and a catheter extended-tip segment 20 distal to the balloon 17. In this example, the catheter segment proximal to the balloon also includes a hub end 11, comprising at least one hub adapter 12 for a guidewire lumen and another hub adapter 14 for inflation of the balloon 17. The catheter 10 also comprises a distal end hole 22, and in this example the catheter 10 is shown with a guidewire entering its hub end 11 through hub adapter 12 and said guidewire 13 exiting the catheter at the distal end hole 11. In this embodiment, the proximal catheter segment 15 may extend from 20 cm to 240 cm in length proximal to the balloon 17, and the distal catheter extended-tip segment 20 may extend from 2 cm to 75 cm distal to the angioplasty balloon 17.

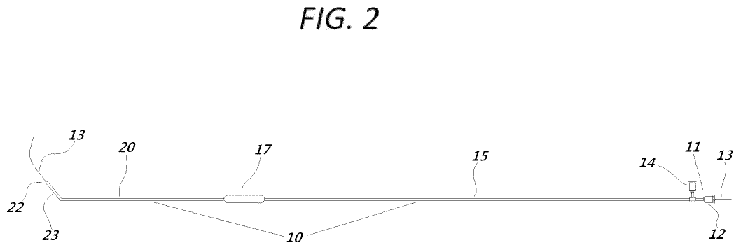

In FIG. 2 another embodiment of a means of reducing catheter exchanges during performance of a percutaneous portosystemic shunt procedure comprising an extended-tip angioplasty balloon catheter 10, in this example the catheter extended-tip segment 20 has an angled segment 23 at its distal end, such that the long axis of the angled segment 23 is not coaxial with the long-axis of the distal catheter extended-tip segment 20, but rather is off-axis by at least 5 degrees, and the angled segment 23 is at least 1 cm long and as long as 20 cm.

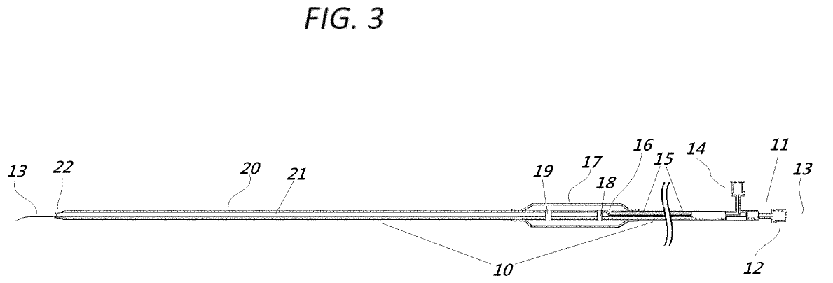

FIG. 3 is a magnified cut-away view of an embodiment of a means of reducing catheter exchanges during performance of a percutaneous portosystemic shunt procedure that comprises an extended-tip angioplasty balloon catheter 10, wherein said extended-tip angioplasty balloon catheter 10 has at least one lumen 21 to accommodate a guidewire 13 or injection of contrast or medication (not shown), said guidewire port configuration comprising either an Over-the-Wire (OTW) or Rapid Exchange (Rx) design, and said guidewire outer diameter (O.D.) comprising a range from 0.009″ to 0.038″. The catheter 10 comprises an angioplasty balloon 17 positioned on a shaft of the catheter, a catheter segment 15 proximal to the balloon 17, and a catheter extended-tip segment 20 distal to the balloon 17. In this example, the catheter segment proximal to the balloon also includes a hub end 11, comprising at least one hub adapter 12 for a guidewire lumen and another hub adapter 14 for inflation of the balloon 17. The extended-tip angioplasty balloon 10 also comprises at least one other lumen 15 for injection of fluid or gas to inflate said angioplasty balloon 17, said other lumen also comprising an exit port 16 extending from the proximal catheter segment 15 into the inside of the angioplasty balloon 17. In this example, the angioplasty balloon 17 contains at least one proximal radiopaque marker band 18 and at least one distal radiopaque marker band 19 affixed to its catheter segment to permit visualization of the angioplasty balloon using fluoroscopy or radiography. The catheter 10 also comprises a distal end hole 22, and in this example the catheter 10 is shown with a guidewire entering its hub end 11 through hub adapter 12 and said guidewire 13 exiting the catheter at the distal end hole 11. In this embodiment, the proximal catheter segment 15 may extend from 20 cm to 240 cm in length proximal to the balloon 17, and the distal catheter extended-tip segment 20 may extend from 3 cm to 75 cm distal to the angioplasty balloon 17.

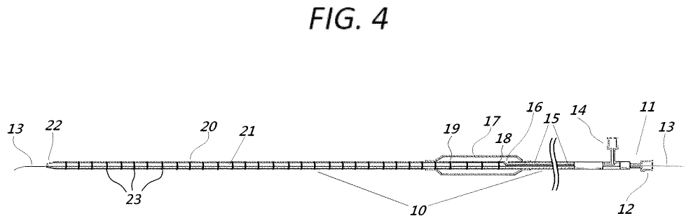

FIG. 4 is a longitudinal view of an exemplary embodiment of a means of reducing catheter exchanges during performance of a percutaneous portosystemic shunt procedure comprising radiopaque marker bands 24 along said extended-tip angioplasty balloon catheter. Those skilled in the art can readily appreciate that said radiopaque marker bands may be located anywhere along said extended-tip angioplasty balloon catheter's length, including on said extended-tip segment, or proximal to said extended tip segment, including within said inflatable member.

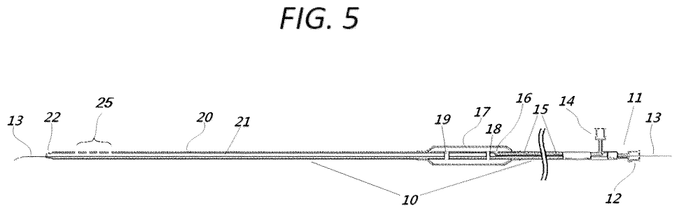

Turning now to FIG. 5, an exemplary embodiment of a means of reducing catheter exchanges during performance of a percutaneous portosystemic shunt procedure that comprises one or more side holes 25 in a distal catheter segment 20, thereby permitting injection of radiopaque contrast or medication distal to the angioplasty balloon around the guidewire 13, without needing to remove the guidewire 13.

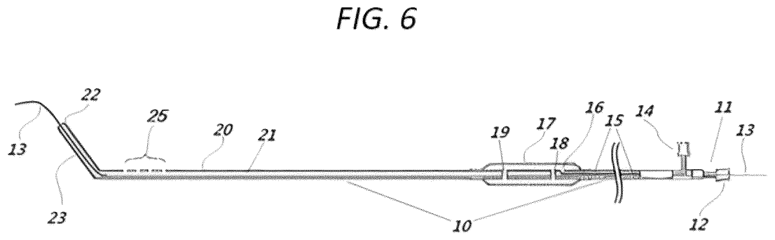

In FIG. 6, an embodiment of a means of reducing catheter exchanges during performance of a percutaneous portosystemic shunt procedure that comprises an extended-tip angioplasty balloon catheter 10 comprising one or more side holes 25 in a distal catheter segment 20, thereby permitting injection of radiopaque contrast or medication distal to the angioplasty balloon around the guidewire 13, further comprising an angled segment 23 at its distal end, such that the long axis of the angled segment 23 is not parallel to the long-axis of the distal catheter extended-tip segment 20, but rather is off-axis by at least 5 degrees, and the angled segment 23 is at least 1 cm long and as long as 20 cm.

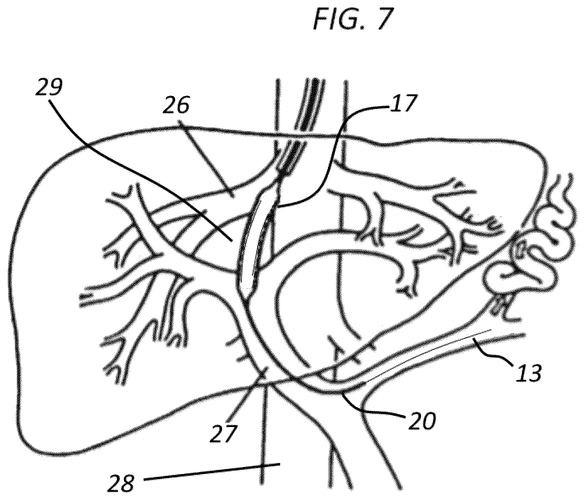

FIG. 7 is an exemplary illustration of a TIPs procedure comprising an extended-tip angioplasty balloon catheter in progress, illustrating aspects of a human anatomy comprising a hepatic vein 26, a portal vein 27, and an inferior vena cava 28, and further comprising illustration of an inflatable member 17 within a hepatic parenchyma 29, an extended-tip segment 20 within a portal vein 27 branch, and a guidewire 13.

### INCORPORATION BY REFERENCE

References and citations to other documents, such as patents, patent applications, provisional patent applications, patent publications, journals, books, papers, web content, that have been made throughout this disclosure are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A method of performing a percutaneous portal to hepatic veno-venous bypass procedure through liver parenchyma comprising the following steps, in this order:
Obtain percutaneous access to the venous vascular system of a patient;
Select a branch of a hepatic vein using a guidesheath, catheter and guidewire in combination;
Replace said catheter with a needle;
Remove said guidewire and advance said needle through said liver parenchyma into a portal vein;
Advance a guidewire through said needle into a portal vein branch;
Remove said needle;
Advance an extended-tip angioplasty balloon catheter through said guidesheath over said guidewire into said portal vein branch so that an extended-tip segment obtains purchase within said portal vein over a secure length of at least 2 cm;
Perform portal venography and portal vein pressure measurements using said extended-tip angioplasty balloon catheter;
Further advance said extended-tip angioplasty balloon catheter so that an inflatable member is positioned within said liver parenchyma;
Expand said inflatable member;
Deflate said inflatable member;
Remove said extended tip angioplasty balloon;
introduce an expandable conduit into said liver parenchyma for preservation of a lumen in said liver parenchyma between said portal vein and said hepatic vein;
Remove sheaths, guidewires, catheters, and angioplasty balloon catheters, from said patient.

2. The method of performing the percutaneous percutaneous portal to hepatic veno-venous bypass procedure through liver parenchyma of claim 1, wherein prior to advancing said extended-tip angioplasty balloon catheter over said guidewire into said portal vein branch said guidesheath in combination with a dilator is advanced into said portal vein over said guide wire.

3. The method of performing the percutaneous portal to hepatic veno-venous bypass procedure through liver parenchyma of claim 1, wherein said extended-tip segment obtains secure purchase within said portal vein of at least 2 cm prior to said angioplasty balloon entering a liver parenchyma.

4. The method of performing the percutaneous portal to hepatic veno-venous bypass procedure through liver parenchyma of claim 1, wherein said guidewire advanced into a portal vein branch is exchanged for a second guidewire after said extended-tip angioplasty balloon catheter is advanced into a position of secure purchase of at least 2 cm within said portal vein.

5. The method of performing the percutaneous portal to hepatic veno-venous bypass procedure through liver parenchymaa TIPS procedure of claim 1, further comprising use of an extended-tip angioplasty balloon catheter comprising radiopaque marker bands to assist an operator's measurement of a tract length to aid selection of said implantable conduit.

6. The method of performing the percutaneous portal to hepatic veno-venous bypass procedure through liver parenchyma of claim 1, wherein after delivery of said conduit within a liver parenchyma is followed by further dilation of said conduit using a catheter comprising an expansile member.

7. The method of performing the percutaneous portal to hepatic veno-venous bypass procedure through liver parenchyma of claim 1, wherein pressure measurements of said hepatic and portal veins are obtained using said extended tip angioplasty balloon catheter.

8. The method of performing the percutaneous portal to hepatic veno-venous bypass procedure through liver parenchyma of claim 1, wherein portal venography is performed using said extended tip angioplasty balloon catheter.

9. A method of performing a percutaneous portal to hepatic veno-venous bypass procedure through liver parenchyma comprising use of a means to obtain secure catheter purchase of at least 2 cm within a portal vein prior to entry within a liver parenchyma of an angioplasty balloon, said means comprising an extended-tip angioplasty balloon catheter.
